(19) United States
(12) Patent Application Publication
(10) Pub. No.: US 2005/0277860 A1
(43) Pub. Date: Dec. 15, 2005

(54) Extended stay-on wound dressing

(75) Inventor: Jarl B. Jensen, Nyack, NY (US)

Correspondence Address: SONNENSCHEIN NATH & ROSENTHAL LLP, P.O. BOX 061080, WACKER DRIVE STATION, SEARS TOWER, CHICAGO, IL, 60606-1080, US

(73) Assignee: Jentec, Inc., Northvale, NJ (US)

(21) Appl. No.: 10/867,388

(22) Filed: Jun. 14, 2004

(51) Int. Cl. A61F 13/00; A61F 15/00

(52) U.S. Cl. 602/41; 602/54

(57) ABSTRACT

A wound dressing having a dressing layer covered by protective cover layer and a release sheet. The dressing has an adhesive composition on a skin-contacting surface. The adhesive composition comprises a highly water-absorbable material (e.g. hydrocolloid), an acrylic adhesive and a tackifier. The composition provides extended stay-on capabilities. The composition also provides an adhesive that is removed from the skin with little pain to the on the skin.

### A. FIELD OF THE INVENTION

The present invention relates to the field of dressings that are applied to the human skin, and more particularly, to the field of dressings that function as fixative and/or protective (or preventive) barriers on the user's skin that may be used while the user is engaged in physical activity.

### B. DESCRIPTION OF RELATED ART

The human skin may be subject to certain types of injuries or conditions that may not limit the user's mobility. A sports enthusiast that enjoys sports such as basketball or tennis may be able to continue to play in a sporting event despite having sustained a cut, a blister or even while having a corn. Even minor surgical incisions, or minor cuts may not be sufficient to limit normal physical activity. Wound dressings are often applied with the expectation that they will stay on for up to several days. However, such dressings may be subject to stresses imposed by the wearer's activity that actually limit the stay-on time dramatically. The dressing may tend to peel off the skin as the wearer moves and the dressing rubs against the wearer's clothing.

Adhesives may be used in order to improve wear-time. However, such adhesives may irritate the skin causing pain and discomfort. For example, acrylic adhesives provide a strong bond to the skin, but are not typically used on wound dressings, particularly where the adhesive may come into contact with the wound bed. The pain and discomfort from stronger adhesives may be further aggravated during removal by the resulting irritation of the skin or by the removal of hair trapped between the skin and the bandage.

It would be desirable to have a dressing that may be used on the human skin for treating or preventing the formation of blisters, corns, warts, calluses, and any cut or wound that may be worn for an extended period of time, even withstanding patient mobility, without causing pain and discomfort during removal. Patients that require the use of fixed therapeutic devices (such as ostomy bags, catheters, or syringes, for example) would also benefit from the use of a fixative dressing that would allow the wearer to move and to take part in some physical activity without becoming susceptible to falling off. Since the fixative dressing would require changing from time to time, it would be advantageous to both extend the wear time to reduce the amount of changing and to ease the pain of removal.

### BRIEF DESCRIPTION OF THE DRAWINGS

Presently preferred embodiments of the invention are described below in conjunction with the appended drawing figures, wherein like reference numerals refer to like elements in the various figures, and wherein:

FIG. 1 represents a perspective view of a dressing according to a preferred embodiment of the present invention.

FIG. 2 represents a fragmentary cross-sectional view of the device in FIG. 1.

FIG. 3 represents a top view of a dressing according to a preferred embodiment of the present invention configured for use as a fixation device.

### DETAILED DESCRIPTION OF THE PREFERRED EMBODIMENT

FIG. 1 shows an individual dressing 5 according to a preferred embodiment of the present invention. This dressing 5 has a thick portion 6 and a thin flange portion 7. The dressing 5 comprises a formulation that combines a hydrocolloid adhesive with acrylic to advantageously provide extended stay-on without the pain and irritation typically experienced with strong adhesives such as pure acrylic adhesives. In addition, the dressing 5 in FIG. 1 may be removed easily without the pain associated with the removal of dressings that use strong adhesives.

The dressing 5 may be used to treat wounds or cuts on the human skin. The dressing 5 may also be used to treat or prevent the formation of blisters, corns, calluses, small cuts, warts, or other such conditions on the human skin. The shape of the dressing 5 in FIG. 1 is rectangular, however, the dressing 5 may have any shape.

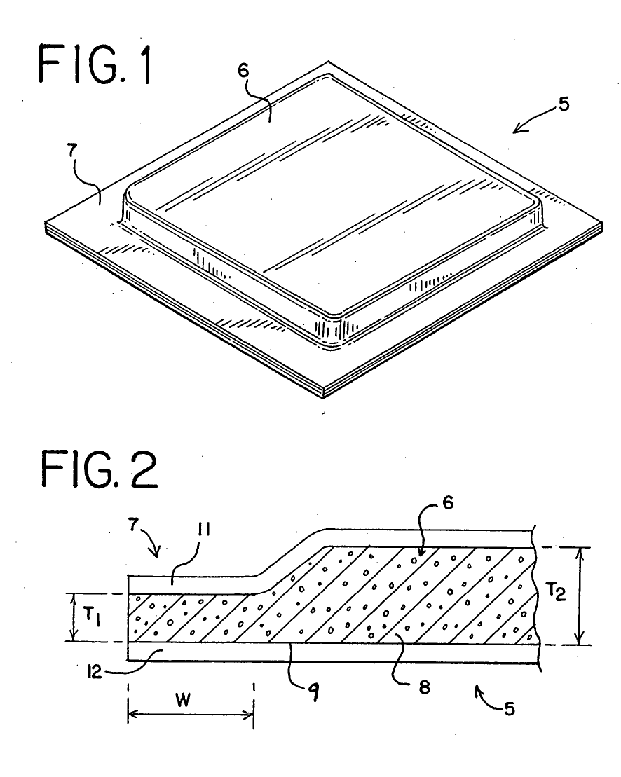

FIG. 2 shows a fragmentary cross-sectional view of one example of the dressing 5 shown in FIG. 1. The dressing 5 in FIG. 2 includes a dressing layer 8, a release sheet 12, and a protective cover layer 11. The dressing layer 8 preferably makes contact with an area of skin at a skin-contacting surface 9 and protects the skin from abrasion and exposure to infectious particles. In exemplary embodiments, the dressing 5 stays on the wound area despite the continued mobility of the patient leading to extended stay-on time. The material properties of the dressing 5 in exemplary embodiments provide unexpected stay-on times combined with a lower degree of pain and discomfort upon the removal of the dressing. The combined features of extended stay-on times and low degree of pain on removal are particularly advantageous when the dressing is applied to areas having an appreciable amount of hair.

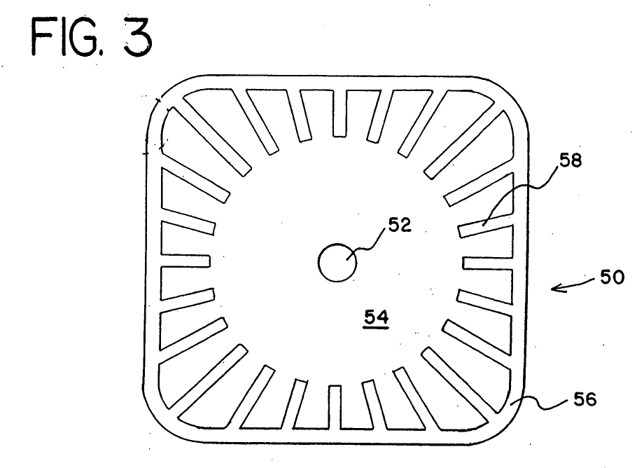

Referring to FIG. 3, exemplary embodiments of the present invention may also include dressings such as a fixation device 50. The fixation device 50 is an example of an ostomy wafer. The fixation device 50 comprises a thick portion 54, a thin portion 56 and a hole 52 that may be used to hold an ostomy device to an opening to the body. The thick and think portions 54, 56 comprise a dressing layer similar to the dressing layer 8 described above with reference to FIG. 2. The composition of the dressing layer of the fixation device of FIG. 3 is described below in conjunction with the description of the dressing layer 8 of FIG. 2. One of ordinary skill in the art will appreciate that the ostomy wafer depicted as the fixation device 5 is only one example of such fixation devices. Other examples include a dressings for affixing catheters, syringes, and other therapeutic devices to the body.

The material comprising the dressing layer 8 includes an adhesive to secure the dressing layer 8 to the skin. The adhesive includes a combination of hot melt acrylic adhesive and a tackifier, such as a hydrocarbon resin. This combination of hot melt acrylic and hydrocarbon resin tackifier surprisingly produces an adhesive that not only stays adhered to skin for an extended amount of time, it also comes off the skin without causing the pain and irritation normally associated with the removal of wound dressings. This is unexpected because acrylic adhesives are too sticky and therefore not commonly used in wound dressing applications at all. Their strong adhesiveness inflicts too much pain on the user upon removal. For wound dressings that cover a large area of skin and that may need to remain on the wearer for an extended amount of time, such as ostomy applications, the skin irritation caused by acrylic adhesives makes dressings with acrylics unusable.

In accordance with exemplary embodiments, the dressing layer 8 comprises:
1. 20-60% by weight of a highly water-absorbable material.
2. 5-60% by weight hot melt acrylic adhesive.
3. 5-40% by weight tackifier.
4. 5-30% elastomer.
5. 5-30% by weight extender or plasticizer.

In exemplary embodiments, the highly water-absorbable material may be a hydrocolloid material. One example of a hydrocolloid that may be used is calcium carboxymethylcellulose (“CMC”). Others include pectin, gelatin, high molecular weight carbowax, carboxypolymethylene, polyacrylate, polyvinyl alcohol, and polyvinyl pyrrolidone.

In exemplary embodiments, the tackifier is preferably a hydrocarbon resin. The elastomer is preferably a styrene-olefin-styrene compound, but may also be polyisobutylene, natural rubber, silicone rubber, arcylonitrile rubber, and polyurethane rubber. The extender is preferably paraffin oil. The extender may also be a material that functions as a plasticizer, particularly in combination with the elastomer. Such plasticizers include glycerin (glycerol), sorbitol, triethylene glycol. The extender may also be mineral oil.

In one preferred embodiment, the dressing layer 8 comprises:
1. 50.5% CMC
2. 13.5% acrylic
3. 17% hydrocarbon tackifier (preferably Arkon P115™)
4. 12% elastomer, preferably Krayton
5. 8% extender, preferably paraffin oil.

For over-the-counter devices, the above quantities are preferably changed to 20% acrylic, 20% hydrocarbon tackifier, and 38% CMC. This combination provides a more tacky compound that would be better suited for high friction environments encountered with over-the-counter devices.

Although not necessary for purposes of the invention, the adhesives (i.e. the hydrocarbon tackifier and the acrylic) should optimally be combined such that the hydrocarbon tackifier is in equal strength per gram weight as the acrylic. Arkon P115™ and acrylic have similar tack properties per density and are therefore mixed in the same percentages. Mixing in the same percentages is not necessary, however, as different hydrocarbon tackifiers may be added to acrylic to provide the advantages of extended stay-on and relatively pain-free removal.

Exemplary embodiments of the present invention have been described. Persons of ordinary skill in the art will appreciate that variations may be made without departure from the scope and spirit of the invention. This true scope and spirit is defined by the appended claims, interpreted in light of the foregoing.

1. A wound dressing comprising a dressing layer having a skin-contacting surface, the improvement comprising:
the dressing layer comprising:
20-60% by weight of a highly water-absorbable material;
5-60% by weight hot melt acrylic adhesive;
5-40% by weight tackifier;
5-30% elastomers;
5-30% by weight extender.

2. The dressing of claim 1 wherein the highly water-absorbable material is a hydrocolloid.

3. The dressing of claim 2 wherein the hydrocolloid is a material selected from the group consisting of: calcium carboxymethylcellulose (“CMC”), pectin, gelatin, high molecular weight carbowax, carboxypolymethylene.

4. The dressing of claim 1 wherein the highly water-absorbable material is a material selected from the group consisting of: polyacrylate, polyvinyl alcohol, polyvinyl pyrrolidone.

5. The dressing of claim 1 wherein the extender is paraffin oil.

6. The dressing of claim 1 wherein the extender is a material that functions as a plasticizer in combination with the elastomer.

7. The dressing of claim 1 wherein the tackifier is a hydrocarbon-based resin.

8. The dressing of claim 1 wherein the elastomer is a composition selected from the group consisting of Styrene-Olefin-Styrene, polyisobutylene, natural rubber, silicone rubber, arcylonitrile rubber, and polyurethane rubber.

9. The dressing of claim 1 wherein the dressing layer comprises:
50.5% CMC;
13.5% acrylic adhesive;
17% tackifier;
12% elastomer; and
8% extender.

10. The dressing claim 1 wherein the dressing later comprises:
38% CMC;
20% acrylic adhesive; and
20% tackifier.

11. The wound dressing of claim 1 further comprising:
a release sheet, and
a protective cover layer.

12. The wound dressing of claim 1 wherein the dressing layer has an inner hole.

13. A composition for application to wounds in a wound dressing comprising:
20-60% by weight of a highly water-absorbable material;
5-60% by weight hot melt acrylic adhesive;
5-40% by weight tackifier;
5-30% elastomers;
5-30% by weight extender.

14. The composition of claim 13 wherein the highly water-absorbable material is a hydrocolloid.

15. The composition of claim 14 wherein the hydrocolloid is a material selected from the group consisting of: calcium carboxymethylcellulose (“CMC”), pectin, gelatin, high molecular weight carbowax, carboxypolymethylene.

16. The composition of claim 13 wherein the highly water-absorbable material is a material selected from the group consisting of: polyacrylate, polyvinyl alcohol, polyvinyl pyrrolidone.

17. The composition of claim 13 wherein the extender is paraffin oil.

18. The composition of claim 13 wherein the extender is a material that functions as a plasticizer in combination with the elastomer.

19. The dressing of claim 13 wherein the tackifier is a hydrocarbon-based resin.

20. The dressing of claim 13 wherein the elastomer is a composition selected from the group consisting of Styrene-Olefin-Styrene, polyisobutylene, natural rubber, silicone rubber, arcylonitrile rubber, and polyurethane rubber.

21. A fixation device having an adhesive composition comprising:
20-60% by weight of a highly water-absorbable material;
5-60% by weight hot melt acrylic adhesive;
5-40% by weight tackifier;
5-30% elastomers;
5-30% by weight extender.

22. The fixation device of claim 21 wherein the highly water-absorbable material is a hydrocolloid.

23. The fixation device of claim 22 wherein the hydrocolloid is a material selected from the group consisting of: calcium carboxymethylcellulose (“CMC”), pectin, gelatin, high molecular weight carbowax, carboxypolymethylene.

24. The fixation device of claim 21 wherein the highly water-absorbable material is a material selected from the group consisting of: polyacrylate, polyvinyl alcohol, polyvinyl pyrrolidone.

25. The fixation device of claim 21 wherein the extender is paraffin oil.

26. The fixation device of claim 21 wherein the extender is a material that functions as a plasticizer in combination with the elastomer.

27. The fixation device of claim 21 wherein the tackifier is a hydrocarbon-based resin.

28. The fixation device of claim 21 wherein the elastomer is a composition selected from the group consisting of Styrene-Olefin-Styrene, polyisobutylene, natural rubber, silicone rubber, arcylonitrile rubber, and polyurethane rubber.

29. The fixation device of claim 21 wherein the dressing layer comprises:
50.5% CMC;
13.5% acrylic adhesive;
17% tackifier;
12% elastomer; and
8% extender.

30. The fixation device claim 21 wherein the dressing layer comprises:
38% CMC;
20% acrylic adhesive; and
20% tackifier.

31. The fixation device of claim 21 further comprising:
a release sheet, and
a protective cover layer.
